SEQUENCE LISTING (16 sequences)

SEQ ID NO: 1 - protein, 155 aa, Homo sapiens
MSESKSGPEYASFFAVMGASAAMVFSALGAAYGTAKSGTGIAAMSVMRPEQIMKSIIPVVMAGIIAIYGLVVAVLIANSLNDDISLYKSFLQLGAGLSVGLSGLAAGFAIGIVGDAGVRGTAQQPRLFVGMILILIFAEVLGLYGLIVALILSTK

SEQ ID NO: 2 - DNA, 25 bp, Artificial sequence
  synthetic construct (primer 16K-3-UP)
cgcgaattcatgtccgagtccaaga

SEQ ID NO: 3 - DNA, 38 bp, Artificial sequence
  synthetic construct (primer 16K+1+DN)
cgggatccctaggacttcatgatctgctccggccgcat

SEQ ID NO: 4 - DNA, 38 bp, Artificial sequence
  synthetic construct (primer 16K+2+UP)
cggaattcatgaagagcggtaccggcattgcggccatg

SEQ ID NO: 5 - DNA, 26 bp, Artificial sequence
  synthetic construct (primer 16K+2+DN)
cgggatccgctcttgtagaggctgat

SEQ ID NO: 6 - DNA, 26 bp, Artificial sequence
  synthetic construct (primer 16K+3+DN)
cgggatccgaatagtcggggctgctg

SEQ ID NO: 7 - DNA, 32 bp, Artificial sequence
  synthetic construct (primer 16K+4+UP)
cggaattcatggacgctggcgtgcggggcacc

SEQ ID NO: 8 - DNA, 24 bp, Artificial sequence
  synthetic construct (primer 16K-3-DN)
cgggatccctttgtggagaggatg

SEQ ID NO: 9 - DNA, 45 bp, Artificial sequence
  synthetic construct (primer TM2-1-1-UP)
aattcatgtccatcatcccagtggtcatggctggcatcatctgag

SEQ ID NO: 10 - DNA, 45 bp, Artificial sequence
  synthetic construct (primer TM2-1-2-DN)
gatcctcagatgatgaaagccatgaccactgggatgatggacatg

SEQ ID NO: 11 - DNA, 17 bp, Artificial sequence
  synthetic construct (primer H16K-UP)
acatgtccgagtccacg

SEQ ID NO: 12 - DNA, 19 bp, Artificial sequence
  synthetic construct (primer H16K-DN)
ctactttgtggagaggatg

SEQ ID NO: 13 - DNA, 23 bp, Artificial sequence
  synthetic construct (primer)
gctctagagaaaatagatgtttg

SEQ ID NO: 14 - DNA, 27 bp, Artificial sequence
  synthetic construct (primer)
ccgctcgagtcactcatacttcggatt

SEQ ID NO: 15 - DNA, 32 bp, Artificial sequence
  synthetic construct (primer 16K+3+UP)
cggaattcatgaactccctgaatgacgacatc

SEQ ID NO: 16 - DNA, 116 bp, Artificial sequence
  synthetic construct (from pXJ41-T7-beta1 ER signal peptide/T7 epitope including Ecor1 and Xba1 restriction sites)
aattcatgaatttgcaactggttttctggattggattgatcagcttgatttgttctgtatttggccaaacagatatggctagcatgactggtggacagcaaatgggttctagaatg

(19) United States
(12) Patent Application Publication
(10) Pub. No.: US 2003/0181369 A1
(43) Pub. Date: Sep. 25, 2003

(54) Method of modulating glycosylation pathways

(76) Inventors: Alan Wildeman, Guelph (CA); Mhairi A. Skinner, Guelph (CA)

Correspondence Address: SUGHRUE MION, PLLC, 2100 PENNSYLVANIA AVENUE, N.W., WASHINGTON, DC, 20037, US

(21) Appl. No.: 10/204,355

(22) PCT Filed: Feb. 26, 2001
(86) PCT No.: PCT/CA01/00211

(51) Int. Cl. A61K 38/17

(52) U.S. Cl. 514/12

(57) ABSTRACT

The invention can be summarized as follows. A method of modulating glycosylation of proteins, in a cell, preferably where the cell is in an animal where the method comprises administering an effective amount of a substance having 16K activity, to an animal in need thereof. The invention relates to a method of modulating tumor-related glycosylation of cell surface receptors and may be used to suppress the invasive growth, migration, or metastasis of tumor cells.

The present invention relates to post-translational modification of proteins. More particularly, the invention relates to the use of 16K or a fragment or a derivative thereof for modifying the glycosylation pattern of proteins.

### BACKGROUND OF THE INVENTION

β1 integrin is a cell surface receptor which binds to the extracellular matrix (fibronectin, laminin, vitronectin) and which plays a role in metastasis of many tumour types. β1 integrin undergoes a glycosylation event in which N-Acetylglucosaminyltransferase V (GlcNAc-TV) initiates tri- and tetra-antennary oligosaccharide branching by adding β1,6 N-Acetylglucosamine (GlcNAc) to terminal mannose residues (Jasiulionis, M. G. et al. (1996)). The epidermal growth factor receptor (EGFR), a cell surface receptor that interacts with epidermal growth factor, is also glycosylated by GlcNAc-TV and has β1,6 GlcNAc branching. In many tumour types there is an increase in β1,6 GlcNAc branching which, in colon carcinomas, serves as a prognostic indicator for tumour recurrence (Fernandes, B., et al. (1991)). Ectopic expression of GlcNAc-TV in epithelial cells has been correlated with cell tumorigenicity and metastasis (Demetriou, M., et al. (1995)).

The GlcNAc-TV enzyme is resident in the Golgi complex of the cell. To date there has been no information about how this enzyme is regulated. Proteins such as β1 integrin are synthesized and inserted into the endoplasmic reticulum, and from there they are shuttled through the early, medial, and trans-Golgi before transport to the surface of the cell.

Both β1 integrin and EGFR are glycosylated by GlcNAc-TV and both have been shown to have significant involvements in cancer cells. β1 integrin is implicated in the invasive processes of many tumor cells (see for example Seftor et al., 1999), and elevated expression of EGER is an indicator of poor prognosis for many cancers, including breast, ovarian and uterine (see for example Kim and Muller, 1999).

Proteins such as β1 integrin and EGFR are synthesized and inserted into the endoplasmic reticulum, and from there they are shuttled through the early, medial, and trans-Golgi before transport to the surface of the cell. The GlcNAc-TV enzyme resides in the Golgi complex of the cell. The vacuolar proton ATPase (V-ATPase) is part of several intracellular membrane compartments including the Golgi complex. V-ATPase directed proton flux is suggested to be involved in transport and processing of cell surface receptors that are shuttled through the Golgi complex (Andresson et al. (1995)). The 16-kDa subunit (16K) of V-ATPase has been shown to interact with β1 integrin (Skinner and Wildeman, 1999). Furthermore, Andresson (Andresson et al., (1995)) discloses that 16K interacts with the E5 oncoprotein of papillomaviruses and inhibits E5 oncoprotein mediated transformation of mouse fibroblast cell lines. A truncated or mutated form of 16K is shown to induce cell transformation. However, there is no teaching that 16K, or a fragment or a mutated form of 16K, has any effect on cell migration, invasive cell growth, or other processes involved in metastasis.

In addition to its involvement in cancer, glycosylation of cell surface receptors effects allergenic resonses and rejection of xenotansplanted organs. In order to advance treatment and management of these diseases it is necessary to identify drugs or other compounds that act on the enzymes that carry out protein glycosylation in cells. Currently, there are no identified natural cellular regulators of glycosylation

It is an object of the invention to overcome disadvantages of the prior art.

The above object is met by the combinations of features of the main claims, the sub-claims disclose further advantageous embodiments of the invention.

### SUMMARY OF THE INVENTION

The present invention relates to post-translational modification of proteins. More particularly, the invention relates to the use of 16K or a fragment or a derivative thereof for modifying the glycosylation pattern of proteins.

The present invention is directed to a method of modulating glycosylation of a protein comprising providing to a cell an effective amount of a substance having the activity of 16K, or a derivative thereof having 16K activity, such that the glycosylation of the protein is modulated. Preferably, the protein is a transmembrane protein.

This invention also pertains to the above method wherein the substance having 16K activity, or a derivative thereof, is selected from the group consisting full length 16K, α 2, a fragment of α 2, α4, a fragment of α 4, α 1,2,3, α 2, α 4, α 1,2, α 2,3, α 2,3,4, α 2 (56-65), α2 (55 to 77), α4 (128 to 149) and a combination thereof.

The present invention also provides a method of modulating glycosylation of a protein comprising providing to a cell an effective amount of a substance having the activity of 16K, or a derivative thereof having 16K activity, such that β1-6 GlcNAc branching of a glycan of the protein is modulated. Preferably, the protein is a transmembrane protein. This invention also pertains to the above method wherein the substance having 16K activity, or a derivative thereof, is selected from the group consisting full length 16K, α 2, a fragment of α2, α 4, a fragment of α4, α 1,2,3, α 2, α 4, α 1,2, α 2,3, α 2,3,4, α 2 (56-65), α2 (55 to 77), α4 (128 to 149) and a combination thereof.

The present invention embraces a method of modulating glycosylation of a protein comprising providing to a cell an effective amount of a substance having the activity of 16K,or a derivative thereof having 16K activity, such that the addition of bisecting GlcNAc residues to a glycan of the protein is modulated Preferably, the protein is a transmembrane protein. This invention also pertains to the method as just described wherein the substance having 16K activity, or a derivative thereof, is selected from the group consisting full length 16K, α 2, a fragment of α 2, α 4, a fragment of α 4, α 1,2,3, α 2, α 4, α 1,2, α2,3, α 2,3,4, α 2 (56-65), α2 (55 to 77), α4 (128 to 149) and a combination thereof.

The present invention also is directed to a method to modulation of glycosylation of a protein comprising:

i) introducing a genetic construct comprising a regulatory sequence operatively linked with a nucleotide sequence encoding 16K, or a derivative of 16K having 16K activity within a cell; and

ii) allowing expression of the nucleotide sequence.

The present invention also provides a method for inhibiting metastasis, comprising, providing an effective amount of 16K or a derivative thereof having 16K activity to a cell.

The present invention pertains to a method for inhibiting metastasis, comprising, expressing a nucleotide sequence encoding 16K or a derivative thereof having 16K activity, within a cell.

The present invention also embraces a method for inhibiting metastasis, comprising administering to an animal in need thereof, an effective amount of a vector, said vector comprising a regulatory sequence operatively linked with a nucleotide sequence encoding 16K or a derivative thereof having 16K activity, and allowing expression of the nucleotide sequence. Also included in the present invention is the method as just described, wherein thel6K or a derivative thereof having 16K activity, is selected from the group consisting of full length 16K, α 2, a fragment of α 2, α 4, a fragment of α 4, α 1,2,3, α 2, α 4, α 1,2, α 2,3, α 2,3,4, α 2 (56-65), α2 (55 to 77), α4 (128 to 149) and a combination thereof.

The present invention also is diretced to a method for inhibiting cell migration, comprising administering to an animal in need thereof, an effective amount of a vector, said vector comprising a regulatory sequence operatively linked with a nucleotide sequence encoding 16K or a derivative thereof having 16K activity, and allowing expression of the nucleotide sequence. This invention also pertains to the method as just defined wherein said method also inhibits invasive cell growth.

The present invention also provides a method for inhibiting invasive cell growth, comprising administering to an animal in need thereof, an effective amount of a vector, said vector comprising a regulatory sequence operatively linked with a nucleotide sequence encoding 16K or a derivative thereof having 16K activity, and allowing expression of the nucleotide sequence. This invention also pertains to the method as just defined wherein said method also inhibits cell migration.

The present invention includes a pharmaceutical composition comprising a vector capable of expressing nucleotide sequence encoding a derivative of 16K, said derivative of 16K selected from the group consisting of α 2, a fragment of α 2, α 4, a fragment of α 4, α 1,2, α 2,3, α 2,3,4, and a combination thereof, within a pharmaceutically acceptable carrier.

The present invention also embraces a nucelotide construct comprising a regulatory sequence operatively linked with a nucleic acid, the nucleic acid comprising a first nucleotide sequence encoding a signal sequence, fused to a second nucleotide sequence encoding an epitope tag, the second nucleotide sequence fused to a third nucleotide sequence encoding a transmembrane protein.

The present invention relates to a method of modulating glycosylation of a protein. The method comprises providing to a cell an effective amount of a substance having the activity of 16K, or a derivative thereof having 16K activity, such that the glycosylation of the protein is modulated.

According to the present invention there is provided a method of modulating glycosylation of a protein. The method comprises providing to a cell an effective amount of 16K, or a fragment or a derivative thereof having 16K activity, such that β1,6 GlcNAc branching of a glycan of the protein is modulated. Preferably, the protein is a transmembrane protein, for example a cell surface receptor. More preferably, the cell surface receptor is an integrin or a growth factor receptor. According to a prefered embodiment of this method the integrin is β1 integrin, the growth factor receptor is EGFR, and the substance is 16K

According to another embodiment of the present invention there is provided a method of modulating glycosylation of a protein comprising providing to a cell an effective amount of a substance having the activity of 16k such that the addition of bisecting GlcNAc residues to a glycan of the protein is modulated, preferably the protein is a transmembrane protein, more preferably the transmembrane protein is an integrin. According to a prefered embodiment of this method the integrin is β1 integrin and the substance is 16K.

In another aspect, the present invention provides a method of treating and preventing cancer. More specifically, the present invention provides a method of inhibiting metastasis. The method comprises providing to a cell an effective amount of 16K such that β1,6 GlcNAc branched, bisecting GlcNAc, or both β1,6 GlcNAc branched, bisecting GlcNAc, of a glycan of an integrin or a growth factor receptor is suppressed.

This summary of the invention does not necessarily describe all necessary features of the invention but that the invention may also reside in a sub-combination of the described features.

### BRIEF DESCRIPTION OF THE DRAWINGS

These and other features of the invention will become more apparent from the following description in which reference is made to the appended drawings wherein:

FIG. 1 shows an outline of asparagine-linked oligosaccharide processing in mammalian cells and characterization of β1 integrin glycosylation. FIG. 1(A) shows a schematic diagram of gylcosylation. FIG. 1(B) shows and characterizes the glycosylation pattern of β1 integrin. Three forms of T7 tagged β1 probed with anti-T7 antibody are shown in lane 1. Treatment with glycopeptidase F (lane 4) and endoH (lane 5) eliminates the middle form (lane 5).

The middle form was preferentially retained on agarose-conjugated conA beads (lane 3). Agarose conjugated L-PHA reacts primarily with the upper form (lane 2).

FIG. 2 shows the effect of 16K on β1,6 branching of β1 integrin. HEK293 cells were co-transfected with a constant amount of T7-β1 integrin and increasing amounts of HSV-tagged 16K. In FIG. 2(A) aliquots of lysates were probed with anti-T7 antibody to indicate the occurrence of three forms of β1 integrin. FIG. 2(B) shows proteins with β1,6 linked oligosaccharides, isolated using agarose-conjugated L-PHA and identified by probing with anti-T7 antibody. Increasing amounts of 16K resulted in the reduction of the largest, L-PHA reactive, form of β1 integrin (lanes 2 to 6). FIG. 2(C) shows the effect of increasing amounts of HSV-tagged 16K on 16K protein levels.

FIG. 3 shows the alteration in the glycosylation of β1 integrin in the presence of full length and truncated versions of 16K. FIG. 3(A) shows the extent of glycosylation of β1 integrin in response to the expression of 16K. Glycosylation was determined using the lectins L-PHA and E-PHA. L-PHA recognizes specifically proteins with β1-6 linked N-actylglucosamines, added by the enzyme GlcNAc-TV, whereas E-PHA recognizes bisecting N-acetylglucosamine residues which are added by GlcNAc-TIII. HEK 293 cells were co-transfected with T7 tagged β1 integrin and increasing amounts of 16K or a mutant of 16K, α1,2,3. RIPA lysates were treated with agarose-conjugated L-PHA and E-PHA and the recovered proteins were probed with anti-T7 antibody to ensure specificity to β1 integrin. Both 16K and α1,2,3 are able to inhibit the addition of bisecting and β1,6 branched GlcNAc residues. FIG. 3(B) shows the extent of glycosylation of β1 integrin in response to the expression of a fragment 16K. Glycosylation was determined as above.

FIG. 4 shows the effect of full length and a truncated form of 16K on cell migration. HEK cells transiently transfected with full length 16K (16K) or a truncated form of 16K (α1,2,3) were examined for their ability to penetrate a Costar Transwell Apparatus coated with 10 μg of laminin, fibronectin, vitronectin or polylysine (control treatment). Control cells were transfected with empty pXJ41 vector (mock transfected).

FIG. 5 shows an illustration of several constructs used in the present invention, as well the effect of these constructs on glycosylation of α1 integrin, FIG. 5(A) shows an illustration of 16K derivatives comprised of specific hydrophobic helices generated with HSV tags. FIG. 5(B) shows the effect of the constructs outlined in FIG. 5(A) on glycosylation. 16K derivatives were cotransfected with T7 tagged β1 integrin into HEK293 cells. Integrin expression was assessed by Western blot analysis of RIPA lysates probed with anti-T7 antibody (panel a), and glycosylation by GlcNAc-TV was assessed by incubating extracts with agarose-conjugated L-PHA and analysing recovered proteins with anti-T7 antibody (panel b). Proteins interacting with 16K or 16K mutants were isolated by immunoprecipitation using anti-HSV antibody followed by agarose conjugated protein A, and recovery of T7-β1 integrin monitored by Western blot analysis using anti-T7 antibody (panel c).

FIG. 6 shows the effect of 16K on β1,6 branching of β1 integrin and EGF-R. HEK293 cells were co-transfected with T7-tagged β1 integrin and EGF-R as well as increasing amounts of HSV-tagged 16K. Lanes 1 to 6 show Western blot analysis of RIPA lysates treated with anti-T7. Lanes 7 to 10 show suppression of β1,6 branching of both β1 integrin (lower band) and the EGF-R (upper band) as detected using L-PHA-conjugated agarose and Western blot analysis with anti-T7 antibody. Lanes 11 to 14 show the same lysates probed with anti-HSV.

FIG. 7 shows the nucleotide construct pXJ41-T7-β1.

### DESCRIPTION OF PREFERRED EMBODIMENT

The present invention relates to methods of modifying the glycosylation pattern of proteins having a role in cancer, autoimmune disease, allergies, asthma, and rejection of xenografts, with the administering of 16K or a fragment or a derivative thereof having 16K activity.

The following description is of a preferred embodiment by way of example only and without limitation to the combination of features necessary for carrying the invention into effect.

The present invention provides a method of modulating glycosylation of proteins, in a cell, including synthetic, altered cells having a golgi complex, preferably where the cell is in an animal. Furthermore, the present invention pertains to the inhibition of metastasis and the control of tumour progression. In one aspect the method comprises administering an effective amount of a substance having 16K activity, to an animal in need thereof.

A “substance having 16K activity” or the “activity of 16K” as used herein is any substance whether a protein, peptide, or a nucleic acid encoding a protein or peptide, having the biological activity of 16K and includes various structural forms of 16K that retain the biological activity of 16K. Examples of a biological activity of 16K, which are not to be considered limiting in any manner include one or more of the following:

alteration of glycosylation;

binding to cell surface receptors for example β1 integrin; or

modulating cell migration.

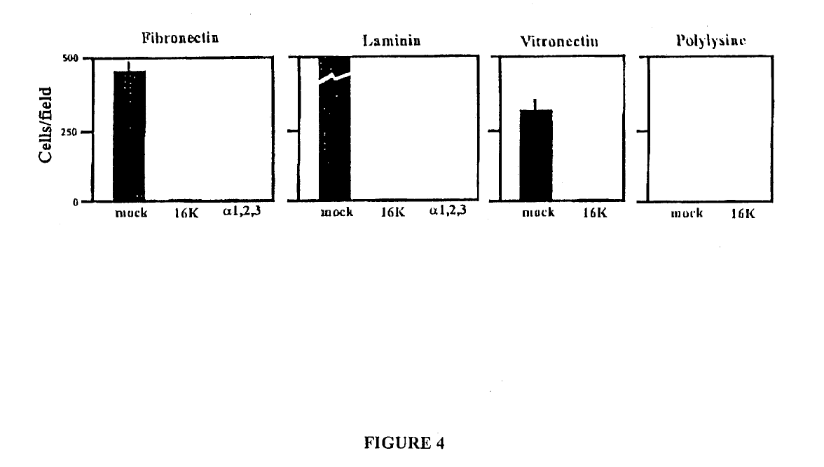

By “metastasis” it is meant, the ability of a cell to migrate and invade tissues, in vivo. Cell migration and invasion can be determined by adsorbing extracellular matrix proteins, for example but not limited to fibronectin, laminin, or vitronectin onto a porous membrane and determining the extent of migration and invasion of a desired cell through the pores coated with matrix (for example, see FIG. 4). Neoplastic transformation is characterized by anchorage independent growth, altered cellular morphology, and growth independent of growth factors that are otherwise needed for regulating normal cell division. Metastasis is characterized with the additional property that neoplastic transformed cells have the ability to migrate on, attach to and invade through epithelia and other tissues and/or extracellular matrices.

Alterations in the post-translational processing of signalling receptor proteins is a feature of neoplastic cell transformation, and a cell surface receptor such as β1 integrin, binds to the extracellular matrix (fibronectin, laminin, vitronectin) and plays a role in metastasis of many tumour types.

16K and Substances Having the Activity of 16K

Within the context of the present invention, a protein of the invention may include various structural forms of 16K which retain the biological activity of 16K. For example, a protein having the activity of 16K may be in the form of acidic or basic salts or in neutral form. In addition, individual amino acid residues may be modified by oxidation or reduction.

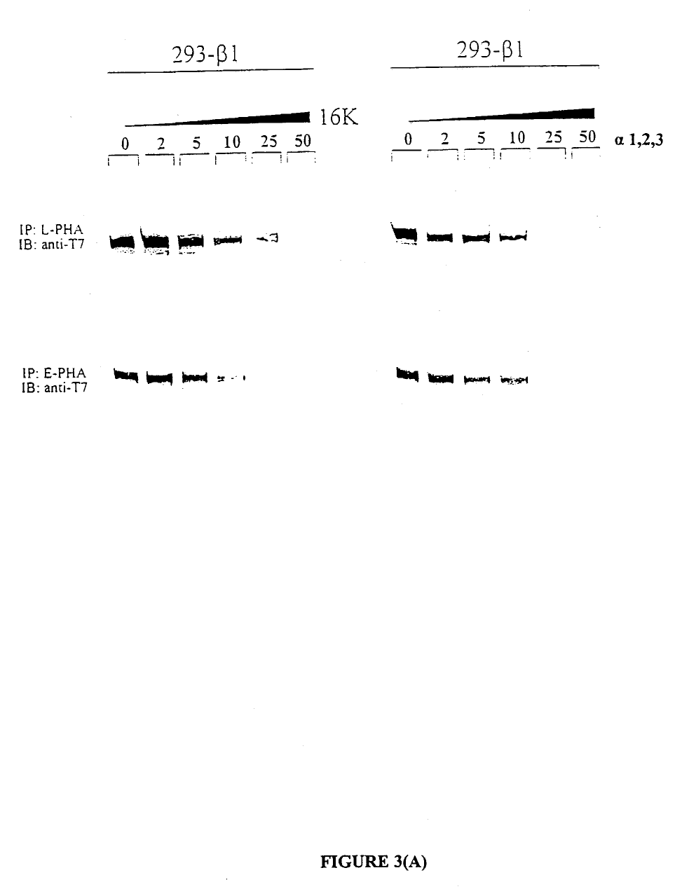

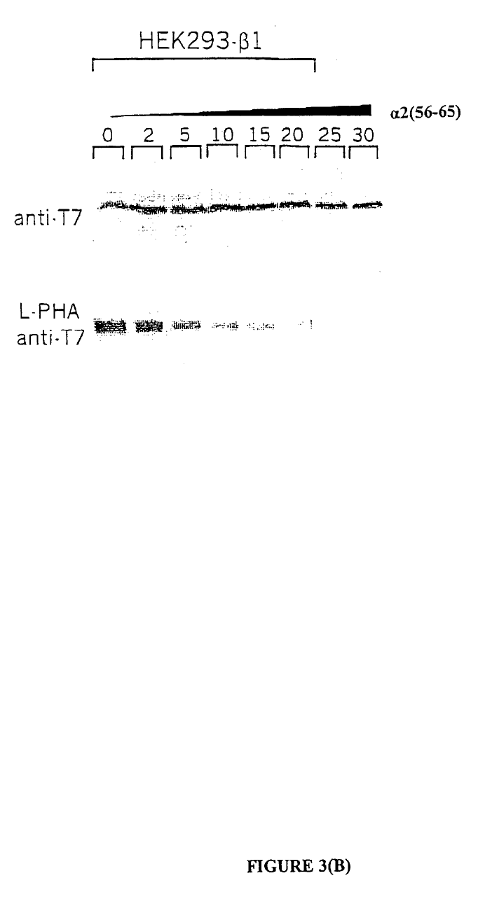

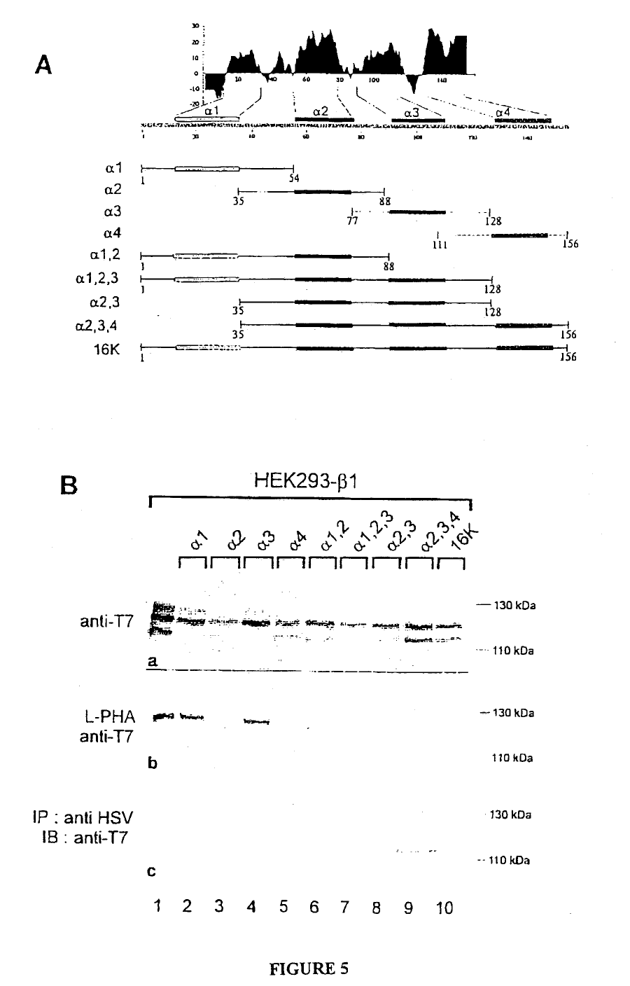

In addition to the full length amino acid sequence of 16K: MSESKSGPEYASFFAVMGASAAMVFSALGAAYGTAKSGTGIAAMSVMRPEQ IMKSIIPVVMAGIIAIYGLVVAVLIANSLNDDISLYKSFLQLGAGLSVGLSGLAA GFAIGIVGDAGVRGTAQQPRLFVGMILILFAEVLGLYGLIVALILSTK (SEQ ID NO:1), the present invention may also include within its scope truncations of a 16K protein, and analogs, and homologs of the protein and truncations thereof as described herein (see Material and Methods of Examples, and FIG. 5). Truncated proteins may comprise peptides of at least ten amino acid residues, although where 16K activity is retained the truncated proteins may be comprised of fewer than ten amino acid residues. Analogs of the protein having the known amino acid sequence and/or truncations thereof as described herein, may include, but are not limited to an amino acid sequence containing one or more amino acid substitutions, insertions, and/or deletions. Amino acid substitutions may be of a conserved or non-conserved nature. Conserved amino acid substitutions involve replacing one or more amino acids of the proteins of the invention with amino acids of similar charge, size, and/or hydrophobicity characteristics. When only conserved substitutions are made the resulting analog should be functionally equivalent. Non-conserved substitutions involve replacing one or more amino acids of the amino acid sequence with one or more amino acids which possess dissimilar charge, size, and/or hydrophobicity characteristics. 16K is composed of four transmembrane helices α 1, α 2, α 3, α 4. Example of fragments or truncated forms of 16K include, but are not limited to α 1, α 2, α 3, α 4, α 1,2, α1,2,3, α 2,3, α 2,3,4 (see for example FIG. 5(A)) and a ten amino acid fragment of α 2 (Ile56-Ile65; see FIG. 3(B)), where α referes to the transmembrane alpha helix of 16K.

One or more amino acid insertions may be introduced into the amino acid sequence of 16K. Amino acid insertions may consist of single amino acid residues or sequential amino acids ranging from 2 to 15 (or more depending upon the activity) amino acids in length. For example, amino acid insertions may be used to destroy target sequences so that the protein is no longer active. This procedure may be used in vivo in circumstances where it is desirable to inhibit the activity of 16K. Deletions may consist of the removal of one or more amino acids, or discrete portions from the amino acid sequence of 16K. The deleted amino acids may or may not be contiguous. The lower limit length of the resulting analog with a deletion mutation is governed by retention of desired activity of the protein.

Analogs of 16K may be prepared by introducing mutations in the nucleotide sequence encoding the protein. Mutations in nucleotide sequences constructed for expression of analogs of a protein of the invention must preserve the reading frame of the coding sequences. Furthermore, the mutations will preferably not create complementary regions that could hybridize to produce secondary mRNA structures, such as loops or hairpins, which could adversely affect translation of the receptor mRNA.

Mutations may be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site specific mutagenesis procedures may be employed to provide an altered gene having particular codons altered according to the substitution, deletion, or insertion required. Deletion or truncation of a 16K protein may also be constructed by utilizing convenient restriction endonuclease sites adjacent to the desired deletion. Subsequent to restriction, overhangs may be filled in, and the DNA religated. Exemplary methods of making the alterations set forth above are disclosed by Sambrook et al (Molecular Cloning: A Laboratory Manual, 2nd Ed., Cold Spring Harbor Laboratory Press, 1989).

The 16K protein of the invention also includes homologs of the amino acid sequence shown in FIG. 5, truncations, or fragments thereof, as described herein. Such homologs are proteins whose amino acid sequences are comprised of amino acid sequences that hybridize under stringent hybridization conditions (as known within the art, for example, as outlined in (Sambrook et al., Molecular Cloning: A Laboratory Manual, 2nd Ed., Cold Spring Harbor Laboratory Press, 1989) with a probe used to obtain a protein of the invention. Homologs of a protein of the invention will have the same regions which are characteristic of the 16K protein.

A homologous protein includes a protein with an amino acid sequence having at least 75%, preferably 80-90% identity with the amino acid sequence of the 16K protein. Such homology determinations may be made using oligonucleotide alignment algorithms for example, but not limited to a BLAST (GenBank URL: www.ncbi.nlm.nih.gov/cgi-bin/BLAST/, using default parameters: Program: blastp; Database: nr; Expect 10; filter: default; Alignment: pairwise; Query genetic Codes: Standard(1)) or FASTA, again using default parameters.

The invention also contemplates isoforms of the 16K proteins. An isoform contains the same number and kinds of amino acids as a protein of the invention, but the isoform has a different molecular structure. The isoforms contemplated by the present invention are those having the same properties as a 16K protein of the invention as described herein.

The proteins of the invention (including truncations, analogs, etc.) may be prepared using recombinant DNA methods. Accordingly, nucleic acid molecules of the present invention having a sequence which encodes a 16K protein may be incorporated according to procedures known in the art into an appropriate expression vector which ensures good expression of the protein. Possible expression vectors include but are not limited to cosmids, plasmids, or modified viruses (e.g., replication defective retroviruses, adenoviruses and adeno-associated viruses), so long as the vector is compatible with the host cell used. The expression “vectors suitable for transformation of a host cell” or “vector”, means that the expression vectors contain a nucleic acid molecule of the invention and regulatory sequences, selected on the basis of the host cells to be used for expression, which are operatively linked to the nucleic acid molecule. “Operatively linked” is intended to mean that the nucleic acid is linked to regulatory sequences in a manner which allows expression of the nucleic acid.

The method of the invention therefore contemplates a recombinant expression vector containing a nucleic acid molecule, or a fragment thereof, and the necessary regulatory sequences for the transcription and translation of a nucleotide sequence encoding the protein-sequence, for example but not limited to 16K, or a derivative of 16K having 16K activity. Suitable regulatory sequences may be derived from a variety of sources, including bacterial, fungal, or viral genes. For example, see the regulatory sequences described in Goeddel, Gene Expression Technology: Methods in Enzymology 185, Academic Press, San Diego, Calif. (1990). Selection of appropriate regulatory sequences is dependent on the host cell chosen, and may be readily accomplished by one of ordinary skill in the art. Examples of such regulatory sequences include: a transcriptional promoter and enhancer or RNA polymerase 15 binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the nucleotide sequence encoding the native protein and/or its flanking regions.

The recombinant expression vectors may also contain genes which encode a fusion moiety which provides increased expression of the recombinant 16K protein; increased solubility of the recombinant protein; and aid in the purification of a target recombinant protein by acting as a ligand in affinity purification. For example, a proteolytic cleavage site may be added to the target recombinant protein to allow eparation of the recombinant protein from the fusion moiety subsequent to purification of the fusion protein.

Recombinant expression vectors can be introduced into host cells to produce a transformed host cell. The term “transformed host cell” is intended to include prokaryotic and eukaryotic cells which have been transformed or transfected with a recombinant expression vector of the invention. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic acid by, for example, electroporation or calcium-chloride mediated transformation. Nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other such laboratory textbooks.

Suitable host cells include a wide variety of prokaryotic and eukaryotic host cells. For example, the proteins of the invention may be expressed in bacterial cells such as E. coli, insect cells (using baculovirus), yeast cells or mammalian cells. Other suitable host cells can be found in Goeddel, Gene Expression Technology: Methods in Enzymology 185, Academic Press, San Diego, Calif. (1991).

The proteins of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

Preparation of a T7 Epitope Tagged Transmembrane Receptor

In order to permit the efficient detection of a desired transmembrane protein, a nucleotide construct was developed comprising a nucleotide sequence encoding an ER signal peptide fused to a transmembrane protein and an epitope tag. This product of this construct allows correct targeting of the transmembrane protein to the ER, and permits detection of the transmembrane protein using the epitope tag and a corresponding anitibody.

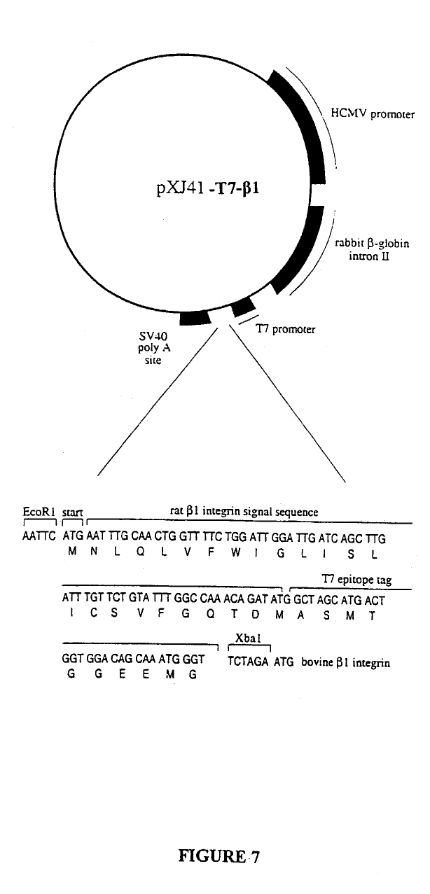

An example of such a vector, which is not not to be considered limiting in any manner, is pXJ41-T7-β1 (see FIG. 7). This nucleotide construct contains at its 5′ end a nucleotide sequence encoding the ER signal peptide followed by nucleotides encoding a T7 epitope, and the full length bovine β1 integrin cDNA. This allows for proper insertion of the receptor into the ER, followed by cleavage of the signal sequence generating an integrin molecule N-terminally tagged with the T7 epitope. This epitope does not interfere with the ability of the integrin receptor to bind extracellular matrix and allows for specific detection of exogenous integrin and mutant integrin proteins. However, it is to be understood that the epitope tag may also be fused to the C terminus of the transmembrane protein as desried.

Therefore the present invention provides a nucleotide construct comprising a regulatory sequence operatively linked with a nucleic acid, the nucleic acid comprising a first nucleotide sequence encoding a ER signal sequence, fused to a second nucleotide sequence encoding a T7 epitope, the second nucleotide sequence fused to a third nucleotide sequence encoding a transmembrane protein.

Effect of 16K on Glycosylation

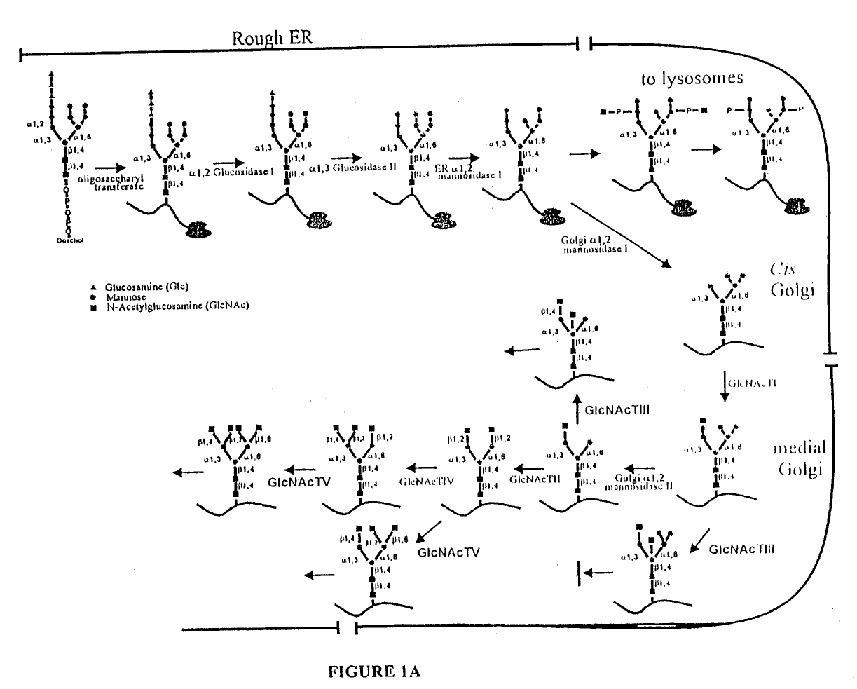

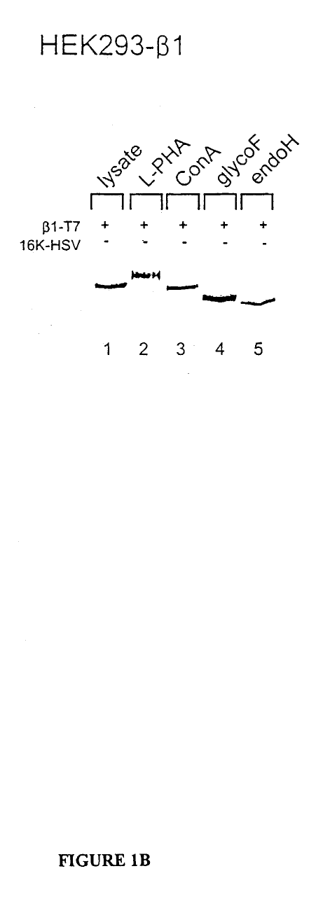

An outline of asparagine-linked oligosaccharide processing in mammalian cells is shown in FIG. 1(A). Glycosylation of integrins occurs co-translationally with the transfer of a dolichol phosphate intermediate to one of 13 potential asparagine residues on the growing integrin polypeptide chain. Carbohydrate units are modified in each of the compartments of the Golgi. Mannose residues are successively trimmed down to the tri-mannosyl core and replaced with N'-acetylglucosamines. The Golgi enzyme GlcNAc-TV catalyzes the addition of β1-6 branched N'-acetylglucosamine (GlcNAc) residues to the β1 integrin precursor generating tri- and tetra-antennary proteins (Dennie et al. (1987)). A fully mature protein is further modified by the addition of galactose and polylactosamine residues.

In the experiments described herein, three major forms of β1 integrin, ranging in size from 110 kDa to 130 kDa are typically produced. However, other forms may exist on this and other cell lines. Of these three forms, only the high molecular weight (130 kDa) has the β1,6 branched GlcNAc residues that are added by GlcNAc-TV in the Golgi complex (FIGS. 1(B) and 2(A), and 6). FIG. 1(A) characterizes the glycosylation pattern of β1 integrin. Concanavalin A (conA) detects molecules with terminal mannose residues that are found predominantly in the ER, and the lectin Phaseolus vulgaris leucoagglutinin (L-PHA) was used to detect the β1,6 branched GlcNAc residues added by GlcNAc-TV. The middle band (ca. 120 kDa) reacts with conA (lane 3) identifying it as the high mannose form present in the ER. Its size was reduced to that of the 110 kDa lower form by endoH (lane 5) and glycopeptidase F (lane 4). The lower form did not react with either conA or L-PHA, identifying it as unglycosylated core protein. The upper form (ca. 130 kDa) contained L-PHA reactive molecules (lane 2), identifying it as the most mature product.

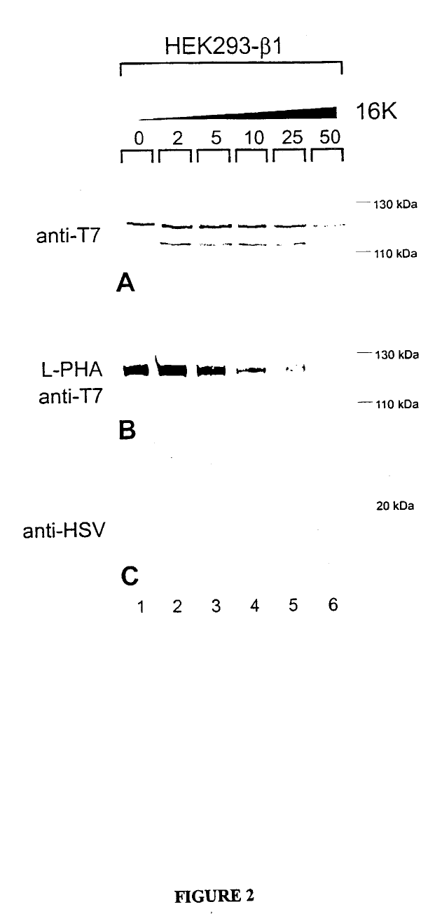

An addition of increasing amounts of DNA encoding 16K results in an corresponding increase in 16K protein (FIG. 2(C)), and inhibits the appearance of the 130 kDa form of the β1 integrin (FIG. 2(A)). As shown in FIG. 2(B), the increased expression of 16K corresponds with a loss of L-PHA reactive β1 integrin, L-PHA specifically recognizes the β1,6-branched form of β1 integrin,

Furthermore, expression of 16K or truncated derivatives of 16K alters the addition of β1-6-branched GlcNAc residues to β1 Integrin. Preferably, derivatives of 16K comprise either the α 2, or a fragment thereof, α 4, or a fragment thereof, or both α 2, or a fragment thereof, and α 4 or a fragment thereof. For example, which is not to be considered limiting in any manner, FIGS. 3(A), (B) and 5 show that the addition of DNA encoding:

16K (amino acids1-156, including the stop codeon);

α 1,2,3 (amino acids 1-128);

α 2 (amino acids 35-88);

α 4 (amino acids 111-156);

α 1,2 (amino acids 1-88);

α 2,3 (amino acids 35-128);

α 2,3,4 (amino acids 35-156);

a 10 amino acid fragment of α 2 (amino acids Ile56-Ile65);

a 23 amino acid fragment of α2 (amino acids 55 to 77); or

a 22 amino acid fragment of α4 (amino acids 128 to 149)

to cells expressing β1 integrin, inhibits the occurrence of β1-6-branched GlcNAc residues to β1 Integrin. Fragments of 16K that lack α 2, or a fragment thereof, α 4, or a fragment thereof, or both α 2, or a fragment thereof, and α 4 or a fragment thereof, are not as effective as derivatives comprised of these fragments, in inhibiting the formation of β1-6-branched GlcNAc residues. No individual helices were observed to form stable interactions with β1 integrin (FIG. 5, panel C, lanes 2-5), suggesting that specific regions of 16K can affect processing of glycosylation residues independently of direct association with β1 integrin.

Furthermore, full length 16K or fragments thereof, for example but not limited to, α 1,2,3 inhibit the occurrence of bisecting GlcNAc residues (FIG. 3(A)).

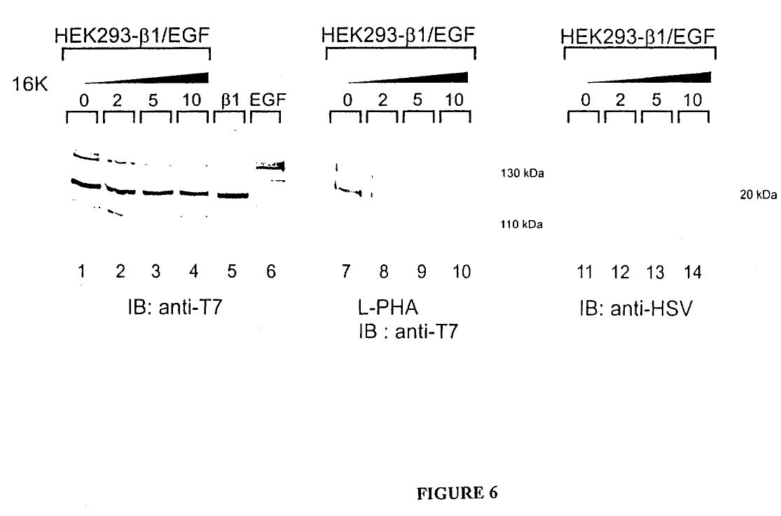

As described herein it is also observed that 16K modulates the glycosylation pattern of other cell surface receptor proteins, including those comprising β1,6 branched oligosaccharides, for example but not limited to epidermal growth factor-receptor (EGF-R). FIG. 6 shows that 16K can alter the glycosylation pattern of EGFR. The addition of increasing amounts of DNA encoding 16K, to cells expressing β1 integrin and EGFR, inhibits the appearance of the L-PHA reactive forms of the EGFR and β1 integrin proteins (lanes 7 to 10).

Therefore, the present invention is directed to the modulation of glycosylation of aprotein comprising providing an effective amount of 16K or a derivative thereof having 16K activity. Preferably, the protein is a transmembrane protein, for example a cell surface receptor. More preferably, the cell surface receptor is an integrin or a growth factor receptor, for example but not limited to β1 integrin, and EGF-R, respectively. Preferably, the 16K is a full length 16K, or a fragment of, α 2, or a fragment thereof, α 4, or a fragment thereof, or both α 2, or a fragment thereof, and α 4 or a fragment thereof, or a fragment selected from the group consisting of α 1,2,3 (amino acids 1-128); α 2 (amino acids 35-88); α 4 (amino acids 111-156); α 1,2 (amino acids 1-88); α 2,3 (amino acids 35-128); α 2,3,4 (amino acids 35-156); a 10 amino acid fragment of α 2 (amino acids 56-65), a 23 amino acid fragment of α2 (amino acids 55 to 77); or a 22 amino acid fragment of α4 (amino acids 128 to 149). Preferably, the 16K, or a derivative thereof having 16K activity, is encoded by a genetic construct capable of synthesizing 16K or a derivative thereof having 16K activity, within the cell.

Addition of 16K or truncated derivatives of 16K to cells suppresses the ability of these cells to migrate and invade. The ability of cells to migrate and invade can be determined using migration/invasion assays, and these assays are indicative of the potential of cells to undergo metastasis (see for example, Praus, M., Wauterickx, K., Collen, D., and Gerard, R. D. 1999. Gene Therapy, 6, 227-236; http://www.ncbi.nlm.nih.gov:80/entrez/query.fcgi?cmd=Retrieve&db=PubMed&list_uids=11182056&dopt=Abstract). In transwell invasion assays (FIG. 4) the expression of full length 16K, and truncated 16K, for example, but not limited to α 1,2,3, abrogated migration of cells through laminin, fibronectin and vitronectin matrices, indicating that 16K, and derivatives thereof, inhibit the invasive abilities of cells. Without wishing to be bound by theory, these data also indicate that 16K mediated loss of β1,6 branching is related to inhibiting the invasive abilities of cells. Furthermore, these data indicate that loss of β1,6 branching in the presence of derivatives of 16K is also related to inhibiting the invasive abilities of cells.

Therefore, the present invention is also directed to a method for inhibiting metastasis, comprising, providing an effective amount of 16K or a derivative thereof having 16K activity. Preferably, the 16K is a full length 16K, or a fragment of, α 2, or a fragment thereof, α4, or a fragment thereof, or both α 2, or a fragment thereof, and α 4 or a fragment thereof, or a fragment selected from the group consisting of α 1,2,3 (amino acids 1-128); α 2 (amino acids 35-88); α 4 (amino acids 111-156); α 1,2 (amino acids 1-88); α 2,3 (amino acids 35-128); α 2,3,4 (amino acids 35-156); a 10 amino acid fragment of α 2 (amino acids 56-65), a 23 amino acid fragment of α2 (amino acids 55 to 77); or a 22 amino acid fragment of α4 (amino acids 128 to 149). More preferably the 16 K is full length 16K, or α 1,2,3. Preferably, the 16K, or a derivative thereof having 16K activity, is encoded by a genetic construct capable of synthesizing 16K or a derivative thereof having 16K activity, within a cell that may develop a metastatic phenotype.

Pharmaceutical Compositions

The present invention provides a composition for use in modulating the glycosylation of a protein in an animal in need thereof comprising an agent which modulates a reaction involved in glycosylation. The agent may be for example: (a) the 16K protein; (b) a vector capable of expressing multiple copies of the 16K gene; or (c) a polypeptide comprising a derivative, fragment or truncation of the 16K protein. It will be readily appreciated that any composition contemplated herein may comprise more than one agent of the invention.

Any or all of the above described substances may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. As used herein the term “animal” includes all members of the animal kingdom.

Administration of an “effective amount” of the pharmaceutical compositions of the present invention is defined as an amount effective, at dosages and for periods of time necessary to achieve the desired result. For example, an effective amount of a substance may vary according to factors such as the disease state, age, sex, and weight of the individual, and the ability of antibody to elicit a desired response in the individual. Dosage regime may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, intranasal, transdermal application, or rectal administration. Depending on the route of administration, the active substance may be coated in a material to protect the compound from the action of enzymes, acids and other natural conditions which may inactivate the compound. If the active substance is a nucleic acid encoding an oligonucleotide it may be delivered using techniques known in the art. Recombinant molecules comprising an oligonucleotide may be directly introduced into cells or tissues in vivo using delivery vehicles such as retroviral vectors, adeno viral vectors and DNA virus vectors. They may also be introduced using physical techniques such as microinjection and electroporation or chemical methods such as co-precipitation and incorporation of DNA into liposomes.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

A composition according to the invention is preferably administered in early stages of cancer development.

Therefore, the present invention also provides for a composition comprising a vector capable of expressing a derivative of 16K selected from the group consisting of α 2, or a fragment thereof, α 4, or a fragment thereof, α 1,2 (amino acids 1-88), α 2,3 (amino acids 35-128), α 2,3,4 (amino acids 35-156) and a combination thereof, within a pharmaceutically acceptable carrier. Preferably, the fragment of α 2 is a 10 amino acid fragment comprising the first 10 amino acids of α 2 (Ile 56 to Ile 65), or a 23 amino acid fragment of α2 (amino acids 55 to 77). Preferably, the fragment of α 4 is a 22 amino acid fragment of α4 (amino acids 128 to 149).

Therapeutic Uses

The present invention provides a method of modulating metastasis comprising administering, to an animal in need thereof, an effective amount of 16K, or a derivative thereof having 16K activity, to modulate transformation and metastasis. Preferably, the 16K, or a derivative thereof having 16K activity, is encoded by a genetic construct capable of synthesizing 16K or a derivative thereof having 16K activity, within a cell that may develop a metastatic phenotype. Furthermore, the present invention pertains to a method for the modulation of metastasis comprising administering to an animal in need thereof, a fragment of 16K having 16K activity. Examples such fragments, which are not to be considered limiting any manner include α 2 (56-65), α 2 (55-77), and α 4 (128-149).

Human xenoreactive antibodies directed against specific carbohydrate groups (found on β1 integrin among other proteins) bind specifically to pig endothelial cells (Holzknect, Z. E., and Platt, J. L. 1995; Holzknect et al., 1999) and are responsible for the rejection of organs in the process of xenotransplantation. 16K may also be useful in reducing the allergenicity of certain foods as allergenic responses are, in part, a result of reactivity to specific glycans present on the proteins found in plants (Garcia-Casado et al., 1996; van Ree et al., 2000).

Therefore, the present invention also provides a method of treatment of allergic responses, asthma, autoimmune disease, or xenograft rejection comprising administering, to an animal in need thereof, an effective amount of 16K or a derivative thereof having 16K activity. Preferably, the 16K, or a derivative thereof having 16K activity, is encoded by a genetic construct capable of synthesizing 16K or a derivative thereof having 16K activity, within a cell that is active in an allergic or asthmatic reaction, or an autoimmune disease, or within a xenotransplant.

In accordance with a preferred embodiment of the present invention, the animal is a human and the 16K blocks complete glycosylation of an integrin protein through inhibition of β1,6 GlcNAc branching, bisecting GlcNAc, or a combination thererof, of a glycan of the protein. It is most preferable to increase the concentration of 16K in the cell and this may be achieved through a variety of approaches.

According to yet another embodiment, the present invention provides a therapeutic agent which acts on the cellular targets of 16K.

The above description is not intended to limit the claimed invention in any manner, furthermore, the discussed combination of features might not be absolutely necessary for the inventive solution.

The present invention will be further illustrated in the following examples. However it is to be understood that these examples are for illustrative purposes only, and should not be used to limit the scope of the present invention in any manner.

### EXAMPLES

Materials and Methods

Cell Culture, Antibodies, and Lectins

Human embryonal kidney cells (HEK293) constitutively expressing T7 polymerase (a gift from Dr. M. A. Billeter, Institut fur Molekularbiologie, Abteilung, Switzerland) were grown in α-MEM in 10% FBS at 37° C. in 5% CO₂. The HSV and T7 antibodies were obtained from Novagen. Agarose-conjugated L-PHA and ConA and alkaline phosphatase conjugated rabbit anti-mouse IgG, were purchased from Sigma.

Assembly of pXJ41-T7-β1

Oligonucleotides encoding the 22 amino acid rat β1 integrin signal sequence (Malek-Hedayat and Rome, 1995) followed by the 11 amino acid T7 epitope (Tsai et al., 1992) were generated, annealed and ligated into the BamHI and EcoRI sites of pXJ41 to make XJ41-T7. These oligos also added an XbaI site upstream of the BamHI site. The assembled full length bovine β1 integrin cDNA (MacLaren and Wildeman, 1995) was inserted into this vector by directionally cloning PCR amplified products using the upstream primer 5′ GCT CTA GAG AAA ATA GAT GTT TG 3′ (SEQ ID NO: 13) and downstream primer 5′ CCG CTC GAG TCA CTC ATA CTT CGG ATT 3′ (SEQ ID NO:14) into the XbaI and XhoI sites of XJ41-T7 (pXJ41-T7-β1; see FIG. 7). The pXJ41-T7-β1 construct contains nucleotides encoding the ER signal peptide followed by nucleotides encoding T7 epitope, and the full length bovine β1 integrin cDNA. This allows for proper insertion of the receptor into the ER, followed by cleavage of the signal sequence generating an integrin molecule N-terminally tagged with the T7 epitope. This epitope does not interfere with the ability of the integrin receptor to bind extracellular matrix and allows for specific detection of exogenous integrin and mutant integrin proteins.

Cloning of Human 16K cDNA and Generation of 16K Mutants

The human 16K cDNA was cloned by RT-PCR from the human pancreatic tumour cell line CRL-80 by Zhao Lu using the primers (H16K-Up) 5′-ACATGTCCGAGTCCACG-3′ (SEQ ID NO:11) and (H16K-DN) 5′-CTACTTTGTGGAGAGGATG-3′ (SEQ ID NO: 12). The HSV-tagged 16K cDNA was made by directionally cloning a PCR product into the EcoR1 and BamH1 sites of the plasmid XJ40-KKO, which adds the HSV tag to the carboxyl terminus of the protein. The 16K PCR fragment was generated using the primers (16K-3-UP) 5′-CGCGAATTCATGTCCGAGTCCAAGA-3′ (SEQ ID NO:2) and (16K-3-DN) 5′-CGGGATCCCTTFGTGGAGAGGAT G-3′ (SEQ ID NO:8). 16K mutants were generated by PCR amplification using primers with appropriate restriction enzymes spanning amino acids identified in FIG. 5 (see below). PCR fragments were HSV epitope tagged at the carboxyl terminus by cloning into pXJ40-KKO (Zuzarte et al., 2000).

Primers used to generate 16K mutants described herein are as follows:

| | | |
|---|---|---|
| 16K | | |
| mutant | Primer | Sequence |

| | | | |
|---|---|---|---|
| α1 | 16K − 3 − UP | 5′ CGCGAATTCATGTCCGAGTCCAAGA 3′ | (SEQ ID NO:2) |
| | 16K + 1 + − DN | 5′ CGGGATCCCTAGGACTTCATGATCTGCTCCGGCCGCAT 3′ | (SEQ ID NO:3) |
| α2 | 16K + 2 + − UP | 5′CGGAATTCATGAAGAGCGGTACCGGCATTGCGGCCATG3′ | (SEQ ID NO:4) |
| | 16K + 2 + DN | 5′ CGGGATCCGCTCTTGTAGAGGCTGAT 3′ | (SEQ ID NO:5) |
| α3 | 16K + 3 + − UP | 5′CGGAATTCATGAACTCCCTGAATGACGACATC3′ | (SEQ ID NO:5) |
| | 16K + 3 + DN | 5′ CGGGATCCGAATAGTCGGGGCTGCTG 3′ | (SEQ ID NO:6) |
| α4 | 16K + 4 + − UP | 5′ CGGAATTCATGGACGCTGGCGTGCGGGGCACC 3′ | (SEQ ID NO:7) |
| | 16K − 3 − DN | 5′ CGGGATCCCTTTGTGGAGAGGATG 3′ | (SEQ ID NO:8) |
| α1,2 | 16K − 3 − UP | (SEQ ID NO:2) | |
| | 16K + 2 + − DN | (SEQ ID NO:5) | |
| α1,2,3 | 16K − 3 − UP | (SEQ ID NO:2) | |
| | 16K + 3 + − DN | (SEQ ID NO:6) | |
| α2,3 | 16K + 2 + − UP | (SEQ ID NO:4) | |
| | 16K + 3 + − DN | (SEQ ID NO:6) | |
| α2,3,4 | 16K + 2 + − UP | (SEQ ID NO:4) | |
| | 16K − 3 − DN | (SEQ ID NO:8) | |
| α2 10aa | TM2 − 1 − 1 − UP | | |
| | 5′AATTCATGTCCATCATCCCAGTGGTCATGGCTGGCATCATCTGAG3′ | (SEQ ID NO:9) | |
| | TM2 − 1 − 2 − DN | | |
| | 5′GATCCTCAGATGATGAAAGCCATGACCACTGGGATGATGGACATG 3′ | (SEQ ID NO:10) | |

Assembly of T7 Tagged EGF Receptor

The EGF-R was cloned into the pXJ41 expression vector and oligonucleotides encoding the 71 amino acid signal sequence (Ulirich et al., 1984) followed by the T7 epitope tag were annealed and ligated upstream of the coding sequence.

Western Blot Analysis

RIPA lysates made from HEK293 cells transiently transfected with pXJ41-T7-β1 were resolved on 8% SDS-PAGE, transferred to nitrocellulose, and probed with anti-T7 antibody. Alternatively, lysates were treated with endoglycosidase H (2 units) or glycopeptidase F and incubated at 37° C. overnight in the manufacturer's (Calbiochem) recommended buffers. Agarose conjugated L-PHA and ConA were used to isolate β1-6 branched and high mannose forms of β1 integrin respectively. Lysates made from cells co-transfected with HSV tagged 16K were treated with anti-HSV antibody and agarose-conjugated protein A and recovered complexes were analysed by Western blot as above. For experiments in which 16K was contransfected with β1 integrin or the EGF-R, total amounts of DNA were made equal by the addition of empty parental pXJ41 vector.

Migration/Invasion Assays

Invasion assays (Albini et al., 1987) were performed in Costar transwell chambers onto which 10 μg of bovine plasma fibronectin, laminin, or polylysine (Sigma) was adsorbed. Prior to adding the cells, the protein matrix was rehydrated for two hours with α-MEM without serum. 2×10⁴ HEK293 cells transiently transfected with 16K were added to the top chamber and allowed to penetrate the matrix for 22 hours at 37° C. The chambers were then washed 3 times with PBS, and any remaining cells were removed from the top surface using a cotton swab. Cells that had penetrated the membrane and reached the lower surface were detected by Giemsa staining and counted. Control cells transfected with the empty pXJ41 vector (mock transfected) were able to invade all matrices tested except a matrix treated with the synthetic polypeptide, polylysine (control treatment).

### Example 1

### 16K Alters the Processing of β1 Integrin

Three major forms of β1 integrin are produced in cells transfected with pXJ41-T7-β1 alone. When this plasmid was cotransfected with one encoding full length or truncated HSV-epitope tagged 16K the relative proportions of these forms varied strikingly (see FIGS. 2(A), 5(B) panel a, and FIG. 6).

16K and β1 integrin were tagged with HSV and T7 epitopes, respectively, to permit specific detection following transfection into HEK 293 cells. Concanavalin A (conA) was used to detect molecules with terminal mannose residues that are found predominantly in the ER, and the lectin Phaseolus vulgaris leucoagglutinin (L-PHA) was used to detect the β1,6 branched GlcNAc residues added by GlcNAc-TV. The T7-epitope tagged β1 integrin produced three main products (FIG. 1(B)) that were identical in size to endogenous forms of β1 integrin (not shown). The middle band (ca. 120 kDa) was reactive with conA (lane 3) and sensitive to endoH glycosidase (lane 5), identifying it as the high mannose form present in the ER. Its size was reduced to that of the 110 kDa lower form by endoH (lane 5) and glycopeptidase F (lane 4). The lower form did not react with either conA or L-PHA, identifying it as unglycosylated core protein. The upper form (ca. 130 kDa) contained L-PHA reactive molecules (lane 2), identifying it as the most mature product. Treatment with brefeldin A, which blocks transport from the ER to the Golgi, inhibited the appearance of only the uppermost band, confirming that the intermediate and low molecular weight forms were ER intermediates (not shown).

### Example 2

### 16K Expression Alters The Addition Of β1-6-branched N-linked Oligosaccharides and Bisecting GlcNAc Residues to the β1 Integrin Molecule

Integrins are extensively glycosylated. In order to determine the effect of 16K expression on the type of β1 integrin processing, two specific glycosylation events were examined. After N-linked glycosoylation in the ER, a portion of the added oligosaccharides serve as a substrate for:

β1-6 N-acetylglucosaminyltransferase V (GlcNAc-TV), a Golgi N-linked oligosaccharide processing enzyme (Chammas et al., 1993; Zheng et al., 1994; Demetriou et al., 1995; Jasiulionis et al., 1996). The lectin leukoagglutinin from Phaseolus vulgaris (L-PHA) recognizes β1-6-branched N-linked oligosaccharides added by GlcNAc-TV; and

bisecting GlcNAc residues added by GlcNAc TIII, are recognized by erythroagglutinin from Phaseolus vulgaris (E-PHA), (Dennis et al., 1987; Jasiulionis et al., 1996).

Agarose-conjugatedL-PHA and E-PHA were used to isolate proteins carrying β1-6-branched oligosaccharides and bisected oligosaccharides from lysates of 293 cells that had been transiently transfected with epitope-tagged wild type β1 integrin, either alone or in combination with increasing amounts of 16K or the 16K mutant lacking the fourth transmembrane helix (α1,2,3). β1 integrin specificity was confirmed by Western blot analysis with anti-T7 antibody. In cells expressing only tagged β1 integrin the predominant L-PHA AND E-PHA reactive species was the larger form of β1 integrin (approx. 130-135 kDa). The medium-sized form was weakly reactive. As may be seen in FIG. 3(A), both L-PHA and E-PHA reactivity on β1 integrin is proportionately reduced with increasing amounts of 16K. The addition of β1-6-branched, or bisecting, oligosaccharides to β1 integrin is also inhibited upon co-expression of α 1,2,3 suggesting that GlcNAc-TV and GlcNac-TIII are somehow regulated by 16K, but not-through the fourth transmembrane helix which binds β1 integrin.

### Example 3

### Expression of 16K, and α 1,2,3, Inhibit β1,6 Branching of β1 Integrin Which Correlates with a Loss of Migratory Abilities of HEK293 Cells.

Increasing amounts of the vector encoding HSV-tagged 16K were co-transfected with a constant amount of T7-tagged β1 integrin. As more 16K was made (FIG. 2(C)), the largest form of the integrin disappeared (FIG. 2(A)). This corresponded with a loss of L-PHA reactive β1 integrin molecules (FIG. 2(B)). There was not a simultaneous build-up of ER-resident forms of the integrin, suggesting that the decrease was due to an alteration in β1 integrin processing rather than on retention of the integrin in the ER. Similarly, increasing amounts of the vector encoding HSV-tagged α 1,2,3 were co-transfected with a constant amount of T7-tagged β1 integrin. As more α 1,2,3 was made a loss of L-PHA and E-PHA reactive β1 integrin (FIG. 2(B), and (FIG. 3(A), respectively) was observed.

Furthermore, in transwell invasion assays (FIG. 4) expression of 16K, or α 1,2,3 abrogated migration through laminin, fibronectin and vitronectin matrices, suggesting a correlation between the loss of β1,6 branching, bisecting or both β1,6 branching and bisecting activities, and the invasive abilities of cells.

### Example 4

### Derivatives of 16K Modulate Glycosylation

A series of HSV-epitope tagged truncated 16K molecules (FIG. 5) was made, and their ability to bind β1 integrin in coimmunoprecipitation experiments compared with their ability to promote the loss of β1,6 branching. The interaction of 16K with β1 integrin required the region of the protein spanning helices 2 to 4 (α 2 and α 4), with helix 4 contributing most to the interaction (FIG. 5B, panel c).

No individual helices formed stable interactions with β1 integrin. Helices 2 and 4 suppressed β1,6 branching as effectively as full length 16K (FIG. 5(B), lanes 3, 5 and 10), while helices 1 and 3 (α 1 and α3) had a reduced effect (lanes 2 and 4). Helix 2 was less active when present with helix 1 (lane 6), but otherwise all variants tested that had either helix 2 or 4 could suppress L-PHA reactivity (lanes 7, 8 and 9).

These data indicate that specific regions of 16K can affect processing independently of direct association with β1 integrin.

### Example 5

### Glycosylation of EGF-R is Also Inhibited by 16K.

The receptor for the epidermal growth factor (EGF-R) also has β1,6 branched oligosaccharides, and it too was T7 epitope tagged and expressed in HEK 293 cells along with T7-tagged β1 integrin and increasing amounts of HSV-tagged 16K (FIG. 6). β1,6 branched proteins were isolated using L-PHA conjugated agarose, and analysed by Western blots probed with anti-T7 antibody. As with β1 integrin, the L-PHA reactive form of the EGF-R was suppressed by 16K (lanes 7 to 10). Co-immunoprecipitation experiments (not shown) failed to detect any interaction between 16K and the EGF-R, again confirming that modulation of glycosylation by 16K may occur without direct interaction with the glycosylation substrate.

### Example 6

### Addition of Derivatives of 16K Inhibits Cell Migration

Peptides incorporating amino acids 56 to 65 of the α2 helix, amino acids 55 to 77 of the α2 helix, or amino acids 128 to 149 of the α4 helix, of full length 16K are synthesized. The abilities of these peptides to inhibit β1,6 branching of β1 integrin and the EGF receptor and to inhibit invasion of HEK 293 cells is assessed.

Cells transiently transfected with either β1 integrin or EGF receptor were treated with peptides throughout the duration of the transfection (at time 0 and after 12 hours). β1,6 branching is assessed by treating lysates with agarose-conjugated L-PHA followed by Western blot analysis using anti-T7 antibody. Inhibition of invasive ability is assessed using Costar transwell filters coated with 10 μg of laminin or fibronectin. After 24 hours of transfection cells are removed from the culture dishes and transferred to the transwell chambers with fresh peptide added to the growth media. The ability of cells to invade the matrix is assessed after 24 and 48 hours.

These results show that increasing amounts of the 16K derivatives, 56 to 65 of the α2 helix, or 55 to 77 of the α2 helix, or amino acids 128 to 149 of the α4 helix, decreased cell mobility and invasion.

All citations are herein incorporated by reference.

The present invention has been described with regard to preferred embodiments. However, it will be obvious to persons skilled in the art that a number of variations and modifications can be made without departing from the scope of the invention as described herein.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

### FULL CITATIONS FOR REFERENCES REFERRED TO IN THE SPECIFICATION

Albini, A., Aukerman, S. L., Ogle, R. C., Noonan, D. M., Fridman, R., Martin, G. R., and Fidler, I. J. (1989) The in vitro invasiveness and interactions with larninin of K-1735 melanoma cells. Evidence for different laminin-binding affinities in high and low metastatic variatns. Clin. Exp. Metastasis, 7, 437451.

Andresson, T., Sparkowski, J., Goldstein, D. J., and Schlegel, R. (1995) Vacuolar H+-ATPase mutants transform cells and define a binding site for the papillomavirus E5 oncoprotein. J. Biol. Chem. 24, 6830-6837.

Chammas, R., Veiga, S. S., Travassos, L. R., and Brentani, R. R. 1993. Functionally distinct roles for glycosylation of and chains in cell-matrix interactions. Proc. Natl. Acad. Sci. USA 90:1795-1799.

Demetriou, M., Nabi, I. R., Coppolino, M., Dedhar, S. and Dennis, J. W. 1995. Reduced contact-inhibition and substratum adhesion in epithelial cells expressing GlcNAc-transferase V. J. Cell Biol. 130:383-392.

Dennis, J. W. and Laferte, S., 1985. Recognition of asparagine-linked oligosaccharides on murine tumor cells natural killer cells. Cancer Res. 45:6034-6040.

Dennis, J. W., Laferte, S., Waghome, C., Breitmen, M., and Kerbel, R. S. 1987. 1-6 branching of Asn-linked oligosaccharides is directly associated with metastasis. Science 236:582-585.

Fernandes, B., Sagman, U., Auger, M., Demetriou, M. and Dennis, J. W. 1991. β1-6 branched oligosaccharides as a marker of tumor progression in human breast and colon neoplasia. Cancer Res. 51:718-723. Garcia-Casado, Gloria, (1996), Role of complex asparagine-linked glycans in the allergenicity of plant glycoproteins, Glycobiol. 6:471-477

Gillespie, G. A., Somlo, S., Germino, G. G., Weinstat-Saslow, D. and Reeders, S. T. (1991) CpG island in the region of an autosomal dominant polycystic kidney disease locus defines the 5 end of a gene encoding a putative proton channel. Proc. Natl. Acad. Sci. U.S.A. 88 (10), 4289-4293.

Holzknecht, Zoie E.,and Platt, J. L. (1995) Identification of Porcine Endothelial Cell Membrane Antigens Recognized by Human Xenoreactive Natural Antibodies, J. Immunol. 154:4565-4575.

Holzknect et al., 1999 Identification of Antigens on Porcine Pulmonary Microvascular Endothelial Cells Recognized by Human Xenoreactive Natural Antibodies, Lab Invest. 79:763

Jasiulionis, M. G., Chammas, R., Ventura, A. M., Travassos, L. R. and Brentani, R. R. 1996. A6 β1 integrin, a major surface carrier of β1-6 branched oligosaccarides, mediates migration of EJ-ras-transformed fibroblasts on laminin-1 independently of its glycosylation state. Cancer Res. 56:1682-1689.

Kim, H., and Muller, W. J. (1999) The role of the epidermal growth factor receptor family in mammary tumorigenesis and metastasis. Exp. Cell Res., 253, 78-87.

MacLaren, L. A., and Wildeman, A. G. (1995) Fibronectin receptors in preimplantation development: cloning, expression, and localization of the α5 and β1 integrin subunits in bovine trophoblast. Biol. Reprod., 53, 153-165.

Malek-Hedayat, S., and Rome, L. H. (1995) Cloning and sequence of the cDNA encoding the rat oligodendrocyte integrin β1 subunit. Gene. 158, 287-290. Nelson, N. 1992. Structure and function of V-ATPases in endocytic and secretory organelles. J. Exp. Biol. 172:149-153.

Petretti, T., Schulze, B., Schlag, P. M. and Kemmer, W. 1999. Altered mRNA expression of glycosyltransferases in human gastric carcinomas. Biochim. et Biophys. Acta 1428:209-218.

Seelentag, W. K., Li, W. P., Schmitz, S. F., Metzger, U., Aeberhard, P., Heitz, P. U. and Roth, J. 1998. Prognostic value of betal1,6-branched oligosaccharides in human colorectal carcinoma. Cancer Res. 58:5559-5564.

Seberger, R. and Chaney, W. G. 1999. Control of metastasis by Asn-linked bl,6-branched oligosaccharides in mouse mammary cancer cells. Glycogiology 9:235-241.

Seftor, R. E., Seftor, E. A., and Hendrix, M. J. (1999) Molecular roles for integrins in human human melanoma invasion. Cancer Metastasis Rev., 18, 359-375.

Skinner, M. A. and Wildeman, A. G. 1999. β1 integrin binds the 16-kDa subunit of vacuolar H+-ATPase at a site important for human papillomavirus E5 and platelet-derived growth factor signalling. J. Biol. Chem. 274:23119-23127.

Takano, R., Nose, M., Nishihira, T. and Kyogoku, M. 1990. Increase of β1-6 branched oligosaccharides in human esophageal carcinomas invasive against surrounding tissue in vivo and in vitro. Am. J. Pathology 137:1007-1011.

Tsai, D. E., Kenan, D. J., and Keene, J. D. (1992) In vitro selection of an RNA epitope immunologically cross-reactive with a peptide. Proc. Natl. Acad. Sci. USA, 89, 8864-8868.

Ullrich, A., Coussens, L., Hayflick, J. S., Dull, T. J., Gray, A., Tam, A. W., Lee, J., Yarden, Y., Libermann, T. A., and Schlessinger, J. (1984) Human epidermal growth factor receptor cDNA sequence and aberrant expression of the amplified gene in A431 epiderrnoid carcinoma cells. Nature, 309, 418-425.

van Ree, R. (2000) Beta(1,2)-xylose and alpha (1,3)-fucose residues having a strong contribution in IgE binding to plant glycoallergens, J. Biol. Chem. 275:11451-8

Yamashita, K., Tachibana, Y., Ohkura, T. and Kobata, A. 1985. Enzymatic basis for the structural changes of asparagine-linked sugar chains of membrane glycoproteins of baby hamster kidney cells induced by polyoma transformation. J. Biol. Chem. 260:3963-3969.

Zheng, M., Fang, H., and Hakamori, S. 1994. Functional role of N-glycosylation in 51 integrin receptor. De-N-glycosylation induces dissociation or altered association of 5 and 1 subunits and concomitant loss of fibronectin binding activity. J. Biol. Chem. 269:12325-12321.

Zuzarte, P. C., Farrance, I. K., Simpson, P. C., and Wildeman, A. G. (2000) Tumor splice variants of the transcription factor TEF-1 induced by SV40 T-antigen transformation. Biochim. Biophys. Acta., 1517, 82-90.

The embodiments of the invention in which an exclusive property of privilege is claimed are defined as follows:

1. A method of modulating glycosylation of a protein comprising providing to a cell an effective amount of a substance having the activity of 16K, or a derivative thereof having 16K activity, such that the glycosylation of the protein is modulated.

2. A method according to claim 1 wherein the protein is a transmembrane protein.

3. A method according to claim 2 wherein the transmembrane protein is an integrin.

4. A method according to claim 3 wherein the integrin is β1 integrin.

5. A method according to claim 1 wherein the substance is selected from the group consisting of full length 16K, α 2, a fragment of α 2, α4, a fragment of α 4, α 1,2,3, α 2, α 4, α 1,2, α 2,3, α 2,3,4, α 2 (56-65), α2 (55 to 77); α4 (128 to 149), and a combination thereof.

6. A method of modulating glycosylation of a protein comprising providing to a cell an effective amount of a substance having the activity of 16K,or a derivative thereof having 16K activity, such that β1-6 GlcNAc branching of a glycan of the protein is modulated.

7. A method according to claim 6 wherein the protein is a transmembrane protein.

8. A method according to claim 7 wherein the transmembrane protein is an integrin.

9. A method according to claim 8 wherein the integrin is β1 integrin.

10. A method according to claim 6 wherein the substance is selected from the group consisting of full length 16K, α2, a fragment of α 2, α4, a fragment of α 4, α 1,2,3, α 2, α 4, α 1,2, α 2,3, α 2,3,4, α 2 (56-65), α2 (55 to 77); α4 (128 to 149), and a combination thereof.

11. A method of modulating glycosylation of a protein comprising providing to a cell an effective amount of a substance having the activity of 16K, or a derivative thereof having 16K activity, such that the addition of bisecting GlcNAc residues to a glycan of the protein is modulated

12. A method according to claim 11 wherein the protein is a transmembrane protein.

13. A method according to claim 12 wherein the transmembrane protein is an integrin.

14. A method according to claim 13 wherein the integrin is β1 integrin.

15. A method according to claim 11 wherein the substance is selected from the group consisting of full length 16K, α 2, a fragment of α 2, α 4, a fragment of α 4, α 1,2,3, α 2, α 4, α 1,2, α 2,3, α 2,3,4, α 2 (56-65) α2 (55 to 77); α4 (128 to 149), and a combination thereof.

16. A method according to claim 1 wherein the cell is a cancer cell in a human.

17. A method of modulating glycosylation of a protein comprising expressing an effective amount of 16K or a derivative thereof having 16K activity within a cell.

18. The method of claim 17, wherein the protein is a transmembrane protein.

19. A method according to claim 18 wherein the transmembrane protein is an integrin.

20. A method according to claim 19 wherein the integrin is β1 integrin.

21. A method according to claim 17 wherein the substance is selected from the group comprising full length 16K, α 2, a fragment of α 2, α 4, a fragment of α 4, α1,2,3, α 2, α 4, α 1,2, α 2,3, α 2,3,4, α 2 (56-65), α2 (55 to 77), α4 (128 to 149), and a combination thereof.

22. A method according to claim 17 wherein the cell is a cancer cell in a human.

23. A method to modulation of glycosylation of a protein comprising:
i) introducing a genetic construct comprising a regulatory sequence operatively linked with a nucleotide sequence encoding 16K, or a derivative of 16K having 16K activity within a cell; and
ii) allowing expression of said nucleotide sequence.

24. A method for inhibiting metastasis, comprising, providing an effective amount of 16K or a derivative thereof having 16K activity to a cell.

25. A method for inhibiting metastasis, comprising, expressing a nucleotide sequence encoding 16K or a derivative thereof having 16K activity, within a cell.

26. A method for inhibiting metastasis, comprising administering to an animal in need thereof, an effective amount of a vector, said vector comprising a regulatory sequence operatively linked with a nucleotide sequence encoding 16K or a derivative thereof having 16K activity, and allowing expression of said nucleotide sequence.

27. The method of claim 26, wherein said 16K or a derivative thereof having 16K activity, is selected from the group consisting of full length 16K, α 2, a fragment of α 2, α 4, a fragment of α 4, α 1,2,3, α 2, α 4, α 1,2, α 2,3, α 2,3,4, α 2 (56-65), α2 (55 to 77), α4 (128 to 149), and a combination thereof.

28. A method for inhibiting cell migration, comprising administering to an animal in need thereof, an effective amount of a vector, said vector comprising a regulatory sequence operatively linked with a nucleotide sequence encoding 16K, or a derivative thereof having 16K activity, and allowing expression of said nucleotide sequence.

29. The method of claim 28, wherein said method also inhibits invasive cell growth.

30 A method for inhibiting invasive cell growth, comprising administering to an animal in need thereof, an effective amount of a vector, said vector comprising a regulatory sequence operatively linked with a nucleotide sequence encoding 16K or a derivative thereof having 16K activity, and allowing expression of said nucleotide sequence.

31. The method of claim 30, wherein said method also inhibits cell migration.

32. A pharmaceutical composition comprising a vector capable of expressing nucleotide sequence encoding a derivative of 16K, said derivative of 16K selected from the group consisting of α 2, a fragment of α 2, α 4, a fragment of α 4, α 1,2, α 2,3, α 2,3,4, and a combination thereof, within a pharmaceutically acceptable carrier.

33. A nucleotide construct comprising a regulatory sequence operatively linked with a nucleic acid, said nucleic acid comprising a first nucleotide sequence encoding a signal sequence, fused to a second nucleotide sequence encoding an epitope tag, said second nucleotide sequence fused to a third nucleotide sequence encoding a transmembrane protein.

34. The nucelotide construct of claim 33, wherein said second nucleotide sequence encoding said epitope tag is upstream of said third nucleotide sequence encoding a transmembrane protein.

35 The nucelotide construct of claim 33, wherein said second nucleotide sequence encoding said epitope tag epitope is downstream of said third nucleotide sequence encoding a transmembrane protein.

36. The nucelotide construct of claim 33, wherein said third nucleotide sequence encoding a transmembrane protein, encodes β1 integrin.

37. The nucelotide construct of cliam 33, wherein said first nucleotide sequence encoding a signal sequence, encodes a β1 integrin signal sequence.

38. The nucelotide construct of cliam 33, wherein said second nucleotide sequence encoding an epitope tag, encodes a T7 epitope.

39. A method according to claim 1 wherein the cell is involved in an allergic reaction.

40. A method according to claim 1 wherein the cell is a involved in an asthmatic reaction.

41. A method according to claim 1 wherein the cell is involved in an autoimmune disease.

42. A method according to claim 1 wherein the cell is within a xenograft.

43. A method according to claim 2 wherein the transmembrane protein is a growth factor receptor.

44. A method according to claim 43 wherein the growth factor receptor is epidermal growth factor receptor.

45. A method according to claim 7 wherein the transmembrane protein is a growth factor receptor.

46. A method according to claim 45 wherein the growth factor receptor is epidermal growth factor receptor.

47. A method according to claim 18 wherein the transmembrane protein is a growth factor receptor.

48. A method according to claim 47 wherein the growth factor receptor is epidermal growth factor receptor.
